(19) United States
(12) Patent Application Publication
(10) Pub. No.: US 2006/0252733 A1
(43) Pub. Date: Nov. 9, 2006

(54) Betulin, betulin derivatives, betulinic acid and betulinic acid derivatives as novel therapeutics in the treatment of disease of lipid and/or glucose metabolism

(75) Inventor: Burkhard Jansen, Pasadena, CA (US)

Correspondence Address: FULBRIGHT & JAWORSKI L.L.P., 600 CONGRESS AVE., SUITE 2400, AUSTIN, TX, 78701, US

(73) Assignee: Novelix Pharmaceuticals, Inc.

(21) Appl. No.: 11/400,779

(22) Filed: Apr. 7, 2006

Related U.S. Application Data
(60) Provisional application No. 60/669,158, filed on Apr. 7, 2005.

(51) Int. Cl. A61K 31/56

(52) U.S. Cl. 514/169

(57) ABSTRACT

The invention relates to method of modulating lipid metabolism comprising contacting a cell with betulin, a betulin derivative, betulinic acid, a betulinic acid derivative, or a related steroid-like compound. In some aspects, the invention relates to the treatment of disorders of lipid metabolism. The invention further relates to methods of identifying compounds that modulate lipid metabolism. The invention also relates to the use of betulin, a betulin derivative, betulinic acid, a betulinic acid derivative, or a related steroid-like compound for preparation of a medicament for the treatment of a disorder of lipid metabolism.

The present application claims the benefit of U.S. Provisional Application Ser. No. 60/669,158 filed Apr. 7, 2005, the entirety of which is incorporated by reference.

### BACKGROUND OF THE INVENTION

1. Field of the Invention

The present invention relates generally to the treatment of metabolic diseases. More specifically, the invention relates to the use of betulin, betulin derivatives, betulinic acid, betulinic acid derivatives and related (steroid-like) compounds either alone or in combination with other medicaments in the treatment of disorders affecting directly or indirectly lipid transport and metabolism.

2. Description of Related Art

Betulinic acid was originally identified as a highly selective inhibitor of human melanoma growth and reported to induce apoptosis in these cells. Since then, a panel of additional cell types were identified which are also responsive against betulinic acid. Additional potential fields of application of this group of substances are disease states like HIV, bacterial infections and inflammatory diseases. The exact mode of action of betulinic acid is still unclear and the binding sites/proteins of betulinic acid have not been identified.

### SUMMARY OF THE INVENTION

Betulin, betulin derivatives, betulinic acid, betulinic acid derivatives and related (steroid-like) compounds are highly suitable either alone or in combination with other medicaments in the treatment of disorders affecting directly or indirectly lipid transport and metabolism. Disorders of this kind are usually referred to as disorders of intermediary metabolism. Elevated plasma lipoprotein levels are important clinically because they can cause life-threatening diseases such as atherosclerosis and pancreatitis.

In some general embodiments, the invention relates to methods of modulating lipid metabolism comprising contacting a cell with betulin, a betulin derivative, betulinic acid, a betulinic acid derivative, or a related steroid-like compound, such compounds are defined in detail below. In certain aspects of the invention, the betulinic acid derivative is NVX-207. In some cases, the cell may be in a subject, for example but not limited to a mammal. In some preferred embodiments, the subject is a human, mouse, or rat. In many embodiments, the subject will have or be at risk of developing a disorder of intermediate metabolism, as defined in detail below.

In some embodiments, the invention relates to methods of modulating glucose metabolism comprising contacting a cell with betulin, a betulin derivative, betulinic acid, a betulinic acid derivative, or a related steroid-like compound, such compounds are defined in detail below. In certain aspects of the invention, the betulinic acid derivative is NVX-207. In some cases, the cell may be in a subject, for example but not limited to a mammal. In some preferred embodiments, the subject is a human, mouse, or rat. In many embodiments, the subject will have or be at risk of developing a disorder of intermediate metabolism, as defined in detail below.

In some more specific embodiments, the invention relates to methods of treating a subject having a disorder of intermediate metabolism comprising administering to the subject an effective amount of betulin, a betulin derivative, betulinic acid, a betulinic acid derivative, or a related steroid-like compound. In certain aspects of the invention, the betulinic acid derivative is NVX-207. In some cases, the subject is a human. These methods may further comprise administering a second compound that is useful in the treatment of a disorder of intermediate metabolism. Those of skill in the art will, without undue experimentation, determine appropriate compounds, dosage concentrations, and dosage regimes for the treatment of subjects in view of the disclosure herein.

In certain aspects of the invention, the betulin, a betulin derivative, betulinic acid, a betulinic acid derivative, or a related steroid-like compound is administered to a subject at a dose of between about 0.01 mg/kg to 30 mg/kg, 0.10 mg/kg to 15 mg/kg, 0.25 mg/kg to 5 mg/kg, 0.25 mg/kg to 3 mg/kg, or between about 0.25 mg/kg to 1.50 mg/kg.

Routes of administration of therapeutic compositions are well-known to those in the art. The betulin, betulin derivative, betulinic acid, betulinic acid derivative, or related steroid-like compound may be administered to a subject by a variety of routes including, for example, orally or intravenously.

In certain aspects of the invention, the betulin, betulin derivative, betulinic acid, betulinic acid derivative, or related steroid-like compound may be administered in combination with one or more additional lipid and/or glucose metabolism-altering agents. The combination of compounds may be administered at the same time or sequentially. The combination of compounds may be formulated into a single composition or separate compositions. Fixed-dose combination lipid-altering drugs are currently available such as extended-release niacin/lovastatin. Other potential drug combinations, such as atorvastatin/amlodipine, ezetimibe/simvastatin, atorvastatin/CETP inhibitor, statin/PPAR agonist, extended-release niacin/simvastatin and pravastatin/aspirin are being developed. Lipid-modulating pharmaceuticals may also include anti-obesity agents which could favourably affect lipid levels. Examples of lipid and/or glucose metabolism-altering agents that may be administered with the betulin, betulin derivative, betulinic acid, betulinic acid derivative, or related steroid-like compound include:

1. Statins (HMG CoA reductase inhibitors), such as simvastatin, atorvastatin, and others;

2. Bile acid sequestrants/resins and cholesterol absorption inhibitors such as ezetimibe, plant stanols/sterols, polyphenols, as well as nutraceuticals such as oat bran, psyllium and soy proteins; phytostanol analogues, squalene synthase inhibitors, bile acid transport inhibitors;

3. SREBP cleavage-activating protein (SCAP) activating ligands;

4. Other current agents that affect lipid metabolism include nicotinic acid (niacin), acipimox, high-dose fish oils, antioxidants and policosanol;

5. Microsomal triglyceride transfer protein (MTP) inhibitors, such as rosuvastatin;

6. Acylcoenzyme A: cholesterol acyltransferase (ACAT) inhibitors, gemcabene, lifibrol, pantothenic acid analogues;

7. Nicotinic acid-receptor agonists;

8. Anti-inflammatory agents (such as Lp-PLA(2) antagonists and AGI1067);

9. Agents that affect nuclear receptors including PPAR-alpha and -gamma agonists, as well as dual PPAR-alpha/gamma and ‘pan’ PPAR-alpha/gamma/delta agonists. This class of compounds encompasses, amongst others, synthetic ligands, such as the thiazolidinediones (TZD), which are used as insulin sensitizers in the treatment of type 2 diabetes;

10. Agents targeting liver X receptor (LXR), farnesoid X receptor (FXR) and sterol-regulatory element binding protein (SREBP);

11. Agents affecting high density lipoprotein cholesterol (HDL-C) blood levels or flux;

12. Cholesteryl ester transfer protein (CETP) inhibitors (such as torcetrapib);

13. CETP vaccines;

14. Upregulators of ATP-binding cassette transporter (ABC) A1, lecithin cholesterol acyltransferase (LCAT) and scavenger receptor class B Type 1 (SRB1);

15. Synthetic apolipoprotein (Apo)E-related peptides;

Examples of lipid and/or glucose metabolism-altering agents can be found, for example, in the following documents, the contents of which are incorporated herein by reference (Berg et al., 2002; Bays and Stein, 2003; Memon et al., 2000; Rieusset et al., 2002; Myerson et al., 2005; Shepherd et al., 2005; Gaofu et al., 2005; Grand-Perret et al., 2001; Chong et al., 2006; Kastelein, 2003; Sudhop and von Bergmann, 2002.

The invention also relates to methods of identifying compounds that modulate lipid metabolism comprising: obtaining a test compound; and determining whether the test compound has an ability to modulate lipid metabolism in a cell. Those of skill in the art are well-versed in a wide variety of screening assays that can be used in the context of the invention and will be able to select and employ appropriate screening assays without undue experimentation in view of the instant disclosure. In some specific embodiments of the invention, the test compound is betulin, a betulin derivative, betulinic acid, a betulinic acid derivative, or a related steroid-like compound. In some cases, the method of determining is high-throughput screening. The cell may be comprised in a cell culture. Alternatively, the cell may be comprised in a subject. In some cases, the subject is preferably a mammal, for example, but not limited to, a human. In some specific embodiments, the practice of these methods will result in the identification of a compound that modulates lipid metabolism, but the mere running of the assay is of value. In some cases, the methods will comprise manufacturing an identified compound. The methods may further comprise administering the manufactured compound to a subject having a disorder of intermediate metabolism.

The invention also relates to the use of betulin, a betulin derivative, betulinic acid, a betulinic acid derivative, or a related steroid-like compound for preparation of a medicament for the treatment of a disorder of lipid metabolism.

As used herein the specification, “a” or “an” may mean one or more. As used herein in the claim(s), when used in conjunction with the word “comprising”, the words “a” or “an” means one or more than one. As used herein “another” may mean at least a second or more.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Chemical structures of betulin, betulinic acid, NVX-207, and LY295427.

FIG. 2A and FIG. 2B: FIGS. 2A and 2B illustrate a method for the identification of NVX-207 binding proteins. FIG. 2A indicates the primary amine group of NVX-207, which allows for coupling of agents with amine reactive groups. As shown in step I of FIG. 2B, the amine group of NVX-207 is modified via the Sulfo-NHS moiety of Sulfo-SBED. This step leading to derivatized NVX-207 is performed by incubating the compound in the dark with a protein lysate containing putative “prey” proteins or with a purified putative “prey” protein. In step II, the bait and prey complex is then captured or trapped by exposing the sample to high-intensity UV light, which activates the phenylazide moiety of Sulfo-SBED. This photoreactive group covalently links to the bound prey protein, capturing the interacting complex. Upon reduction of this complex (step III), for example by boiling under reducing conditions in SDS-PAGE protein sample buffer, cleavage of the disulfide bond occurs. The biotin label that first resided with the bait remains bound to the prey protein (step 1V) and serves to detect and/or purify the protein of interest.

FIG. 3A and FIG. 3B: MALDI-TOF-MS (Matrix-Assisted-Laser-Desorption/Ionization-Time-Of-Flight-Mass-Spectrometry) analysis of five NVX-207-binding protein spots isolated from 2-D silver stained gels. The most relevant results of the MS analysis are shown.

### DESCRIPTION OF ILLUSTRATIVE EMBODIMENTS

A. “Betulin,” “Betulin Derivatives,” “Betulinic Acid,” “Betulinic Acid Derivatives,” and “Related Steroid-Like Compounds.”

The expressions “betulin,” “betulin derivatives,” “betulinic acid,” “betulinic acid derivatives,” and “related steroid-like compounds” refer to tri-terpenoid compounds in which the substituents on the A-, B-, C- and D-rings are modified in a way well known to a person skilled in the art.

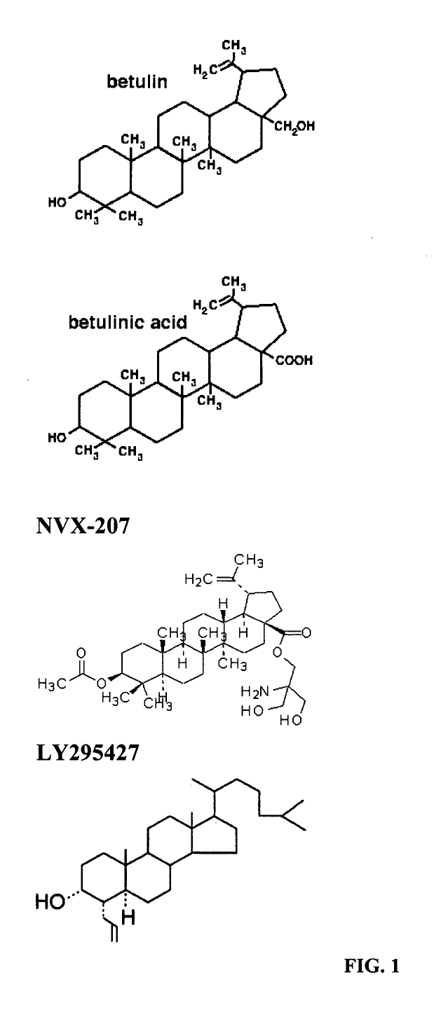

Betulin, which is also known as lup-20(29)-ene-3β,diol, has the molecular formula C₃₀H₅₀O₂. Betulinic acid, which is also known as 3β-Hydroxy-20(29)-lupaene-28-oic acid, has the molecular formula C₃₀H₄₈O₃. Betulin and betulinic acid are commercially available from Sigma-Aldrich Co. NVX-207 is a betulinic acid derivative. The structures of betulin, betulin acid, and NVX-207 are provided in FIG. 1.

In certain aspects of the invention, the betulinic acid derivative has the general formula (I): 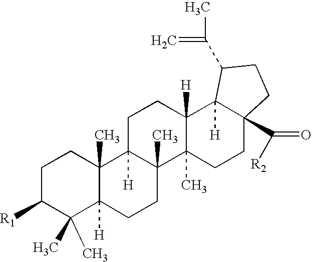
wherein R₁ represents a hydroxy group, an amino group, a protected hydroxy group, or a protected amino group; and R₂ represents:

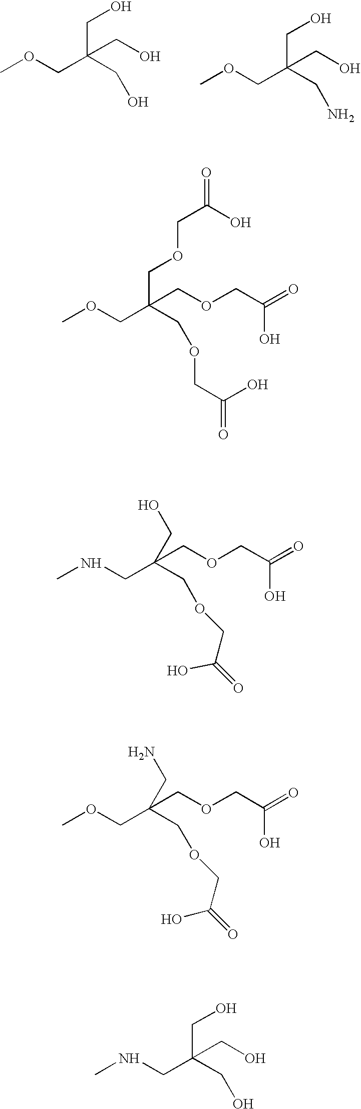

Additional examples of betulin derivatives, betulinic acid derivatives, and related (steroid-like) compounds suitable for use in connection with the present invention can be found, for example, in the following documents, the contents of which are incorporated herein by reference: U.S. Pat. No. 6,403,816; U.S. Pat. No. 6,228,850; U.S. Pat. No. 5,962,527; U.S. Pat. No. 5,869,535; U.S. Pat. No. 6,214,814; U.S. Pat. No. 6,048,847; US Pubn. Appln. 2002068098; US Pubn. Appln. 2002099164; US Publn. Appln. 2002091091; CA 2515384; DE 19854402; EP 22-19990922; JP 19-19970603; JP 17-20010619; JP 12-19970331; JP 7-19991026; JP 13-19981006; JP 7-19970311; WO 95/04526; WO 0209698; WO 99/47113; WO 99/16449; WO 00/03749; WO 00/03748; WO 02/09720; WO 96/29068; WO 00/24762; WO 00/66080; WO 00/66072; WO 00/59492; WO 00/59492; WO 00/26174; WO 01/17497; WO 01/90046; WO 02/26762; WO 02/26761; WO 01/72265; WO 02/09719; WO 9639033; WO 02/16395; WO 98/51294; WO 98/51293; WO 00/46235; WO 01/72315; WO 02/53138; WO 02/05296; WO 02/43736; WO 02/78685; WO 02/78468.

B. Definition of Disorders of Intermediary Metabolism

As set forth above, the current invention relates to the treatment of disorders affecting directly or indirectly lipid transport and metabolism. Disorders of this kind are usually referred to as disorders of intermediary metabolism.

For examples, definitions, and a listing of some members of this class of clinical disorders, including primary and secondary hypercholesteriemias, see Harrison's Principles of Internal Medicine (McGraw Hill, 12^{th} edition, pp. 1814-1825).

Further examples of such disease states include hyperlipoproteinemias and other disorders of lipid metabolism, including: primary hyperlipoproteinemias, such as familial forms of hypercholesterinemia, hypertriglyceridemia or familial combined hyperlipidemia and others.

Also included are clinical disorders associated with secondary hyperlipoproteinemia including endocrine and metabolic diseases, such as diabetes mellitus, lipodystrophies, and others.

Also included are drug-induced disorders of intermediary metabolism, such as those induced by alcohol, contraceptives, and other drugs.

Renal, hepatic, immunologic, and stress-induced disease states involving disorders of intermediate metabolism are also examples of such disorders. The invention has uses in the context of lipodystrophias and other rare disorders.

See, for examples, Harrison's Principles of Internal Medicine (McGraw Hill, 12^{th} edition, pp. 1883-1887).

Finally, the invention has use in regard to generalized lipodystrophia, partial lypodystrophia, and localized dystrophia.

C. Formulations and Administration

Pharmaceutical compositions of the present invention comprise an effective amount of one or more of the betulin, betulin derivative, betulinic acid, betulinic acid derivative, or related steroid-like compound as described by the present invention dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one betulin, betulin derivative, betulinic acid, betulinic acid derivative, or related steroid-like compound will be known to those of skill in the art in light of the present disclosure, and knowledge in the art concerning pharmaceutical compositions as exemplified by Remington's Pharmaceutical Sciences, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, antioxidants, salts, coatings, surfactants, preservatives (e.g., methyl or propyl p-hydroxybenzoate, sorbic acid, antibacterial agents, antifungal agents), isotonic agents, solution retarding agents (e.g. paraffin), absorbents (e.g. kaolin clay, bentonite clay), drug stabilizers (e.g. sodium lauryl sulphate), gels, binders (e.g. syrup, acacia, gelatin, sorbitol, tragacanth, polyvinylpyrrolidinone, carboxy-methyl-cellulose, alginates), excipients (e.g. lactose, milk sugar, polyethylene glycol), disintegration agents (e.g. ager-ager, starch, lactose, calcium phosphate, calcium carbonate, alginic acid, sorbitol, glycine), wetting agents (e.g. cetyl alcohol, glycerol monostearate), lubricants, absorption accelerators (e.g. quaternary ammonium salts), edible oils (e.g. almond oil, coconut oil, oily esters or propylene glycol), sweetening agents, flavoring agents, coloring agents, fillers, (e.g. starch, lactose, sucrose, glucose, mannitol, slilcic acid), tabletting lubricants (e.g. magnesium stearate, starch, glucose, lactose, rice flower, chalk), carriers for inhalation (e.g. hydrocarbon propellants), buffering agents, or such like materials and combinations thereof, as would be known to one of ordinary skill in the art (see, for example, Remington's Pharmaceutical Sciences, 1990). Except insofar as any conventional carrier is incompatible with the active ingredient, its use in the therapeutic or pharmaceutical compositions is contemplated.

In certain embodiments of the invention, the betulin, betulin derivative, betulinic acid, betulinic acid derivative, or related steroid-like compound may be formulated into a composition in a salt form. Pharmaceutically acceptable salts include the acid addition salts, e.g., those formed with the free amino groups of a proteinaceous composition or which are formed with inorganic acids such as for example, hydrochloric, hydrobromic, or phosphoric acids; or such organic acids as acetic, oxalic, tartaric, benzoic, lactic, phosphorific, citric, maleaic, fumaric, succinic, tartaric, napsylic, clavulanic, stearic, or mandelic acid. Salts formed with the free carboxyl groups can also be derived from inorganic bases such as for example, sodium, potassium, ammonium, calcium magnesium or ferric hydroxides; or such organic bases as isopropylamine, trimethylamine, histidine or procaine.

In some embodiments of the invention, the betulin, betulin derivative, betulinic acid, betulinic acid derivative, or related steroid-like compound may be formulated within cyclodextrins or in any other formulation known in the art suitable for use with lipophilic agents.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The betulin, betulin derivative, betulinic acid, betulinic acid derivative, or related steroid-like compound may also comprise different types of carriers depending on whether it is to be administered in solid or liquid form, and whether it need to be sterile for such routes of administration as injection. The present invention can be administered orally, intradermally, subcutaneously, topically, intravenously, or by other methods or any combination of the forgoing methods as would be known to one of ordinary skill in the art (see, for example, Remington's Pharmaceutical Sciences, 1990).

The betulin, betulin derivative, betulinic acid, betulinic acid derivative, or related steroid-like compound when administered orally may be in the form of tablets, capsules, sachets, powders, granules, lozenges, reconstitutable powders, or liquid preparations.

The actual dosage amount of a composition of the present invention administered to a subject can be determined by physical and physiological factors such as body weight, gender, severity of condition, the type of disease being treated, previous or concurrent therapeutic interventions, idiopathy of the patient, time of the administration, rate of excretion of the particular compound, and on the route of administration. The practitioner responsible for administration will, in any event, determine the concentration of active ingredient(s) in a composition and appropriate dose(s) for the individual subject. In certain aspects of the invention, it is contemplated that a subject weighing 70 kg would be given a dose of between about 10 mg to 1000 mg of the betulin, betulin derivative, betulinic acid, betulinic acid derivative, or related steroid-like compound. More preferably, a subject weighing 70 kg would be given a dose of between about 20 mg to 100 mg of the betulin, betulin derivative, betulinic acid, betulinic acid derivative, or related steroid-like compound.

D. Examples

The following examples are included to demonstrate preferred embodiments of the invention. It should be appreciated by those of skill in the art that the techniques disclosed in the example which follows represent techniques discovered by the inventor to function in the practice of the invention, and thus can be considered to constitute specific modes for its practice. However, those of skill in the art should, in light of the present disclosure, appreciate that many changes can be made in the specific embodiments which are disclosed and still obtain a like or similar result without departing from the scope of the invention.

### Example 1

The inventor has identified—by gene chip analysis—the so-called insulin-induced gene 1 (INSIG-1) as a highly induced gene after treatment of a human lung cancer cell line (A549) with NVX-207, a derivative of betulinic acid. In addition to INSIG-1, the expression of several other genes that are known to be involved in lipid and/or glucose metabolism were highly and reproducibly upregulated within several hours after the addition of NVX-207 to the culture medium of the lung cancer cell line.

For global gene expression analysis, total RNA was isolated from sub-confluent cell cultures at 4 and 14 hours after addition of NVX-207. In short, cells were grown under standard cell culture conditions and treated or with or without NVX-207 (in ethanol at 10 mg/ml) at final concentrations of 1.5 (IC50) and 2.0 μg/ml (IC80). For each time point and concentration, respectively, total RNA form 3 equally treated cultures was pooled and used for gene chip analysis. The experiment was repeated once. Four and 14 hours after addition of NVX-207, cells were washed with PBS and lysed directly in the culture dish with RNA-Bee according to the manufacturers instructions. Total RNA was then analyzed by using Affymetrix gene chips according to the manufacturers instructions. A list of the most relevant genes for lipid and/or glucose metabolism that were upregulated by NVX-207 is provided in Table 1.

TABLE 1
NVX-207-Upregulated Genes with a Functional Role in Lipid
and/or Glucose Metabolism

Sterol-C4-methyl oxidase-like
Low density lipoprotein receptor (familial hypercholesterolemia)
Insulin induced gene 1
Isopentenyl-diphosphate delta isomerase
Squalene epoxidase
3-hydroxy-3-methylglutaryl-Coenzyme A reductase
Hydroxysteroid (17-beta) dehydrogenase 7
Farnesyl-diphosphate farnesyltransferase 1
Sterol-C5-desaturase (ERG3 delta-5-desaturase homolog, fungal)-like
3-hydroxy-3-methylglutaryl-Coenzyme A synthase 1 (soluble)
Fatty-acid-Coenzyme A ligase, long-chain 3
Pyruvate dehydrogenase phosphatase isoenzyme 2
Lipin 1
Acetyl-Coenzyme A acetyltransferase
Machado-Joseph disease gene (spinocerebellar ataxia 3)
7-dehydrocholesterol reductase
24-dehydrocholesterol reductase
Stearoyl-CoA desaturase (delta-9-desaturase)
Hydroxysteroid (17-beta) dehydrogenase 7
NAD(P) dependent steroid dehydrogenase-like protein

A list of NVX-207-Regulated genes, including the fold induction for each gene, is provided in Table 2.

TABLE 2
NVX-207-Regulated Genes

| Fold-Induction | Gene |
|---|---|
| 3.36 | Ring finger protein |
| | 125 (T-cell RING |
| | activation protein 1, |
| | TRAC-1) |
| 2.58 | Muscleblind-like |
| | (Drosophila) |
| 2.56 | Insulin induced gene 1 |
| 2.37 | Homo sapiens |
| | mRNA; cDNA |
| | DKFZp686L01105 |
| | (from clone |
| | DKFZp686L01105) |
| 2.26 | Homo sapiens cDNA |
| | FLJ26120 fis, clone |
| | SYN00419 |
| 2.09 | Homo sapiens |
| | mRNA; cDNA |
| | DKFZp686L01105 |
| | (from clone |
| | DKFZp686L01105) |
| 1.89 | Farnesyl-diphosphate |
| | farnesyltransferase 1 |
| 1.88 | Lipin 1 |
| 1.87 | Sterol-C4-methyl |
| | oxidase-like |
| 1.85 | Homo sapiens, clone |
| | IMAGE: 6205812, |
| | mRNA |
| 1.78 | Low density |
| | lipoprotein receptor |
| | (familial |
| | hypercholesterolemia) |
| 1.78 | Isopentenyl- |
| | diphosphate delta |
| | isomerase |
| 1.77 | Homo sapiens, clone |
| | IMAGE: 5314747, |
| | mRNA |
| 1.76 | Solute carrier family |
| | 2 (facilitated glucose |
| | transporter), member 6 |
| 1.73 | Homo sapiens |
| | mRNA; cDNA |
| | DKFZp686L01105 |
| | (from clone |
| | DKFZp686L01105) |
| 1.70 | Thyroid hormone |
| | receptor interactor 11 |
| 1.67 | Solute carrier family |
| | 7, (cationic amino |
| | acid transporter, y+ |
| | system) member 11 |
| 1.63 | 3-hydroxy-3- |
| | methylglutaryl- |
| | Coenzyme A synthase |
| | 1 (soluble) |
| 1.61 | Hypothetical protein |
| | MGC34695 |
| 1.60 | Squalene epoxidase |
| 1.58 | Homo sapiens cDNA |
| | FLJ13272 fis, clone |
| | OVARC1001004. |
| 1.56 | Homo sapiens cDNA |
| | FLJ11590 fis, clone |
| | HEMBA1003758. |
| 1.56 | Chromosome 22 open |
| | reading frame 20 |
| 1.55 | Homo sapiens cDNA |
| | FLJ12932 fis, clone |
| | NT2RP2004897. |
| 1.54 | Hypothetical protein |
| | from BCRA2 region |
| 1.54 | Homo sapiens cDNA |
| | FLJ14096 fis, clone |
| | MAMMA1000752. |
| 1.54 | 7-dehydrocholesterol |
| | reductase |
| 1.52 | Splicing factor, |
| | arginine/serine-rich 1 |
| | (splicing factor 2, |
| | alternate splicing |
| | factor) |
| 1.52 | Homo sapiens cDNA |
| | FLJ14253 fis, clone |
| | OVARC1001376. |
| 1.51 | Squalene epoxidase |
| 1.51 | Regulator of G- |
| | protein signalling 12 |
| 1.51 | NAD(P) dependent |
| | steroid |
| | dehydrogenase-like |
| 1.50 | Zinc finger protein |
| | 160 |
| 1.50 | Hydroxysteroid (17- |
| | beta) dehydrogenase 7 |
| 1.50 | Homo sapiens cDNA |
| | FLJ12075 fis, clone |
| | HEMBB1002425. |
| 1.50 | Adenylate cyclase 9 |
| 1.49 | Hypothetical protein |
| | LOC126661 |
| 1.48 | Homo sapiens |
| | mRNA; cDNA |
| | DKFZp779K0112 |
| | (from clone |
| | DKFZp779K0112) |
| 1.48 | Ankyrin repeat |
| | domain 6 |
| 1.47 | Tumor necrosis factor |
| | (ligand) superfamily, |
| | member 9 |
| 1.46 | Zinc finger protein |
| | 226 |
| 1.46 | Hypothetical protein |
| | PRO1496 |
| 1.46 | Hypothetical protein |
| | MAC30 |
| 1.46 | Homo sapiens |
| | transcribed sequence |
| | with weak similarity |
| | to protein |
| | ref: NP_060265.1 |
| 1.45 | Natural killer-tumor |
| | recognition sequence |
| 1.45 | Machado-Joseph |
| | disease |
| | (spinocerebellar |
| | ataxia 39 |
| 1.45 | Hypothetical protein |
| | FLB8034 PRO2158 |
| 1.44 | Homo sapiens cDNA: |
| | FLJ23159 fis, clone |
| | LNG09628 |
| 1.44 | Acetyl-Coenzyme A |
| | acetyltransferase 2 |
| | (acetoacetyl |
| | Coenzyme A |
| | thiolase) |
| 1.43 | Surfactant, |
| | pulmonary-associated |
| | protein B |
| 1.42 | Mitochondrial |
| | ribosomal protein |
| | L30 |
| 1.41 | Stearoyl-CoA |
| | desaturase (delta-9- |
| | desaturase) |
| 1.41 | Neurolysin |
| | (metallopeptidase M3 |
| | family) |
| 1.41 | Homo sapiens cDNA |
| | FLJ33420 fis, clone |
| | BRACE2020028. |
| 1.40 | Pyruvate |
| | dehydrogenase |
| | phosphatase |
| | isoenzyme 2 |
| | KIAA0912 protein |
| 1.39 | Mucolipin 3 |
| 1.39 | Hypothetical protein |
| | FLJ13611 |
| 1.39 | D15F37 (pseudogene) |
| 1.38 | Ubiquitin specific |
| | protease 6 (Tre-2 |
| | oncogene) |
| 1.38 | KIAA0335 gene |
| | product |
| 1.37 | Sterol-C5-desaturase |
| | (ERG3 delta-5- |
| | desaturase homolog, |
| | fungal)-like |
| 1.37 | Protocadherin 11 Y- |
| | linked |
| 1.35 | Hypothetical protein |
| | FLJ11577 |
| 1.34 | Hypothetical protein |
| | LOC125893 |
| 1.32 | Hypothetical protein |
| | FLJ38499 |
| 1.31 | Fatty-acid-Coenzyme A |
| | ligase, long-chain 3 |
| 1.30 | Heterogeneous nuclear |
| | ribonucleoprotein |
| | A2/B1 |
| 1.28 | Natural killer-tumor |
| | recognition sequence |
| 1.26 | Phorbol-12-myristate- |
| | 13-acetate-induced |
| | protein 1 |
| 1.26 | Hypothetical protein |
| | LOC283680 |
| 1.26 | Homeo box B2 |
| 1.25 | Ubiquitin specific |
| | protease 32 |
| 1.25 | 24-dehydro-cholesterol |
| | reductase |
| 1.24 | Zinc finger protein 430 |
| 1.24 | Hypothetical protein |
| | FLJ14639 |
| 1.23 | Pantothenate kinase 3 |
| 1.23 | KIAA0101 gene |
| | product |
| 1.23 | Jerky homolog-like |
| | (mouse) |
| 1.22 | Homo sapiens |
| | transcribed sequence |
| | with weak similarity to |
| | protein |
| | ref: NP_060265.1 |
| 1.21 | KIAA1450 protein |
| 1.21 | Apoptosis inhibitor 5 |
| 1.20 | SOCS box-containing |
| | WD protein SWiP-1 |
| 1.20 | ALEX3 protein |
| 1.17 | Pinin, desmosome |
| | associated protein |
| 1.16 | Hypothetical protein |
| | FLJ10997 (ZNF654 |
| | zinc finger protein 654 |
| | [Homo sapiens]) |
| 1.16 | Choline kinase |
| 1.14 | Putative translation |
| | initiation factor |
| 1.13 | Homo sapiens mRNA; |
| | cDNA |
| | DKFZp586O031 (from |
| | clone DKFZp586O031) |
| 1.12 | Glyoxalase I |
| 1.10 | SKI-interacting protein |
| 1.10 | Receptor-interacting |
| | factor 1 |
| 0.92 | Haspin |
| 0.89 | Exportin 6 |
| 0.87 | ATP synthase, H+ |
| | transporting, |
| | mitochondrial F0 |
| | complex, subunit s |
| | (factor B) |
| 0.86 | Syntaxin 16 |
| 0.86 | Hypothetical protein |
| | BC008207 |
| 0.86 | 216814_at |
| 0.81 | Homo sapiens cDNA |
| | FLJ46153 fis, clone |
| | TESTI4001037 |
| 0.79 | Mucin 4, |
| | tracheobronchial |
| 0.79 | Cyclic nucleotide gated |
| | channel alpha 1 |
| 0.78 | SP140 nuclear body |
| | protein |
| 0.78 | Phosphodiesterase 4A, |
| | cAMP-specific |
| | (phosphodiesterase E2 |
| | dunce homolog, |
| | Drosophila) |
| 0.77 | Latrophilin 3 |
| 0.73 | Nicotinamide |
| | nucleotide |
| | adenylyltransferase 2 |
| 0.70 | Hypothetical protein |
| | DKFZp761A132 |
| 0.70 | Homo sapiens full |
| | length insert cDNA |
| | clone ZE03A06 |
| 0.69 | SPTF-associated factor |
| | 65 gamma |
| 0.66 | H63 breast cancer |
| | expressed gene |
| 0.66 | DEAD (Asp-Glu-Ala- |
| | Asp) box polypeptide |
| | 54 |
| 0.62 | HMT1 hnRNP |
| | methyltransferase-like 1 |
| 0.60 | Melan-A |

From the gene list presented in Table 1, it is evident that NVX-207 and similar chemical compounds, such as betulin, betulin derivatives, betulinic acid, and betulinic acid derivatives, are ideal candidates for the treatment of disease of lipid and/or glucose metabolism.

NVX-207-induced gene expression analysis demonstrated an astonishing overlap of genes induced by this compound with the gene set induced by another sterol analogue designated LY295427. The chemical structure of LY295427 is provided in FIG. 1, along with the chemical structures of betulin, betulinic acid, and NVX-207. LY295427 was found to reduce plasma cholesterol levels in animals by increasing the expression of hepatic low density lipoprotein (LDL) receptors. Of note, induction of LDL-receptor expression was also one of the major findings of the gene-expression analysis after NVX-207 treatment in the lung cancer cell line. As is described further below, upregulation of LDL-receptor expression is one major strategy to lower plasma cholesterol levels. Micromolar concentrations of LY295427 induced the metabolism of LDL in oxysterol-treated cultured cells and inhibited the stimulation of cholesteryl ester synthesis mediated by oxysterols. Micromolar concentrations were also used in the NVX-207 experiments demonstrating induction of the genes listed in Table 1.

The results of Brown and Goldstein's pioneering studies of familial hypercholesterolemia revealed the physiologic importance of the LDL receptor, as the absence of the LDL Receptor leads to hypercholesteremia and atherosclerosis (Berg et al., 2002). The total concentration of cholesterol and LDL in the plasma is markedly elevated in this genetic disorder, which results from a mutation at a single autosomal locus. In familial hypercholesterolemia, cholesterol is deposited in various tissues because of the high concentration of LDL cholesterol in the plasma. The molecular defect in most cases of familial hypercholesterolemia is an absence or deficiency of functional LDL receptors. Homozygotes have almost no functional receptors for LDL, whereas heterozygotes have about half the normal number. Consequently, the entry of LDL into liver and other cells is impaired, leading to an increased plasma level of LDL. All clinical consequences of an absence or deficiency of the LDL receptor can be attributed to the ensuing elevated level of LDL cholesterol in the blood.

Homozygous familial hypercholesterolemia can be treated only by a liver transplant. A more generally applicable therapy is available for heterozygotes and others with high levels of cholesterol. One important aim is to reduce the amount of cholesterol in the blood by increasing the expression of LDL receptors. As is described above, the Betulinic acid derivative NVX-207 is capable of upregulating LDL receptor expression. One rarely noticed fact is that hydroxymethylglutaryl-coenzyme A reductase inhibitors (statins), which are most widely used in the treatment of lipid disorders, are capable of inducing cell death—in similar concentration ranges compared to betulinic acid and its derivatives—by stimulating apoptosis in various cell types (Demierre M F et al., Nat Rev Cancer, 2005). As is shown in Example 3 below, NVX-207 is capable of reducing simvastatin-induced cell death in a human melanoma cell line. The fact that cytotoxic effects are not additive when combining both classes of compound may be helpful in reducing side effects.

Another gene shown to be upregulated by NVX-207 was fatty-acid-Coenzyme A ligase, long-chain 3. The protein encoded by this gene is an isozyme of the long-chain fatty-acid-coenzyme A ligase family. Although differing in substrate specificity, subcellular localization, and tissue distribution, all isozymes of this family convert free long-chain fatty acids into fatty acyl-CoA esters, and thereby play a key role in lipid biosynthesis and fatty acid degradation.

Furthermore, NVX-207 upregulated pyruvate dehydrogenase phosphatase isoenzyme 2 (PDP2). The pyruvate dehydrogenase complex (PDC) is inactivated in many tissues during starvation and diabetes to conserve three-carbon compounds for gluconeogenesis. Starvation and streptozotocin-induced diabetes cause decreases in PDP2 mRNA abundance, PDP2 protein amount, and PDP activity in rat heart and kidney. Re-feeding and insulin treatment effectively reverses these effects of starvation and diabetes, respectively. NVX-207 also induced the expression of lipin 1. This is of significance since lipin levels in adipose tissue influence the fat storage capacity of the adipocyte, whereas lipin levels in skeletal muscle acted as a determinant of whole-body energy expenditure and fat utilization. Phan and Reue (2005) concluded that variations in lipin levels alone are sufficient to induce extreme states of adiposity and may represent a mechanism by which adipose tissue and skeletal muscle modulate fat mass and energy balance. NVX-207 also induced the expression of 24-dehydrocholesterol reductase. The enzyme 3-beta-hydroxysterol delta-24-reductase (DHCR24), a member of the flavin adenine dinucleotide (FAD)-dependent oxidoreductases, catalyzes the reduction of the delta-24 double bond of sterol intermediates during cholesterol biosynthesis. In addition, induction of NAD(P) dependent steroid dehydrogenase-like protein as well as of hydroxysteroid (17-beta) dehydrogenase 7 by NVX-207, both of which are involved in the sterol biosynthetic pathway, also indicate a role in lipid metabolism.

Apart from the above mentioned lipid and glucose metabolism regulating genes, one of the major findings of the gene chip analysis was that the induction of the insulin-induced gene 1 (INSIG-1) after treatment of human lung cancer cells with NVX-207. INSIG-1 was highly and reproducibly upregulated within several hours after the addition of NVX-207 to the culture medium of the human lung cancer cell line. The observed and unexpected rapid upregulation (up to 3-fold) of INSIG-1 within 4 hours of the addition of NVX-207 is of importance because of its role in the control of lipid synthesis.

INSIG-1 was originally identified using microarray analysis of mRNA from adipose tissue of diabetic rats treated with PPAR-gamma agonists (e.g. Rosigliazone), which act as insulin-sensitizers. It has been suggested that the regulation of INSIG-1 by PPAR-gamma agonists such as Rosiglitazone may couple insulin sensitizers with the regulation of lipid honieostasis. INSIG-1 has been described in the scientific literature to play a substantial role in the control of lipid synthesis of animal cells. INSIGs coordinate lipid synthesis via their sterol-dependent binding to ER membrane proteins. Insulin-induced gene expression has been suggested to restrict lipogenesis and to block differentiation in preadipocytes. In addition, it is claimed in the scientific literature that INSIG-1 is as a key regulator of many important gene products involved in adipocyte recruitment and hyperplasia. Overexpression of INSIG-1 in the livers of transgenic mice reduces insulin-stimulated lipogenesis. Isolation of mutant cells lacking INSIG-1 has provided proof for the pivotal role of INSIG-1 in the control of lipid synthesis in cultured cells.

Taken together, the above mentioned observations made by gene chip analysis of NVX-207-treated cells immediately suggest the potential of the compounds as defined above as medicaments in the treatment of disorders which primarily or secondarily affect lipid metabolism. This notion is also based on the structural similarity of betulinic acid and derivatives (steroid-like compounds) thereof to other compounds in clinical or preclinical use for the treatment of disorders affecting, for example, cholesterol biosynthesis.

### Example 2

It was demonstrated that the betulinic acid derivative, NVX-207, binds to Apolipoprotein A1, a major regulator of lipid metabolism and cholesterol transport. Until now, the identity of betulinic acid binding proteins was completely unknown.

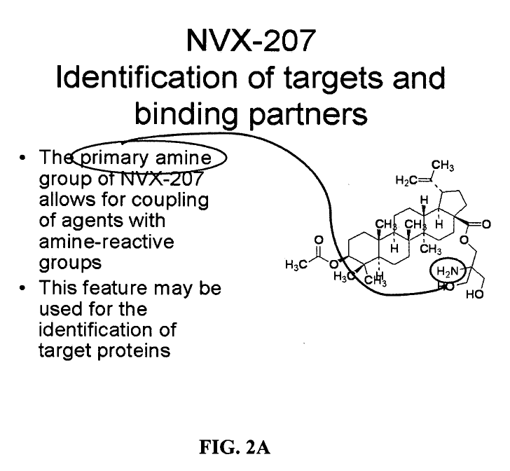

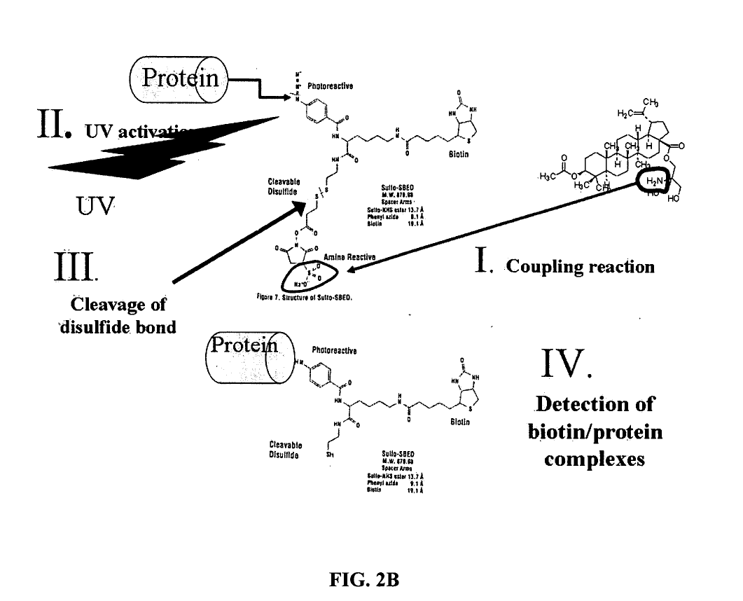

The presence of a primary amine group on NVX-207, a unique feature of this derivative compared to other known betulinic acid derived compounds, allows the coupling of other chemical compounds. A commercially available kit from Pierce was used to couple NVX-207 with biotin (Pierce-Sulfo-BED). This procedure allows the identification of NVX-207 binding proteins in protein lysates from any source. This procedure is generically illustrated in FIG. 2. In short, the amine groups of NVX-207 are modified via the Sulfo-NHS moiety of Sulfo-SBED. This derivatized NVX-207 is then incubated in the dark with a lysate or with a purified putative “prey” protein. The bait and prey complex is then captured or trapped by exposing the sample to high-intensity UV light, which activates the phenylazide moiety of Sulfo-SBED. This photoreactive group covalently links to the bound prey protein, capturing the interacting complex. Upon reduction of this complex, the biotin label that first resided with the bait transfers to the prey protein. The biotin label also functions as the detection target for the NVX-207:prey complex or the prey protein upon Western blot analysis. The resulting biotin derivatized complex or prey protein can be detected using streptavidin-HRP or an anti-biotin antibody and chemiluminescent detection. The biotin label can also be used as a handle to purify the NVX-207:prey complex over an avidin/streptavidin/NeutrAvidin/monomeric avidin biotin-binding protein support.

Using the procedure described above, it was demonstrated that NVX-207 binds to Apolipoprotein A1. Fetal Calf Serum (FCS) was UV-treated with

NVX-207 (at 5 μg/ml final concentration) coupled to SEBD with or without a 20-fold molar excess of unlabeled NVX-207 and resolved by 2-D gel electrophoresis. The addition of an excess of unlabelled NVX-207 is to discriminate between specific and unspecific protein binding of the NVX-207/SBED complex by competitive inhibition. Several spots of similar molecular weight (˜24 kDa) and differing IP (in the range of 4.8 to 5.6) were detected by streptavidin-POD after 2-dimensional (2-D) gel electrophoresis.

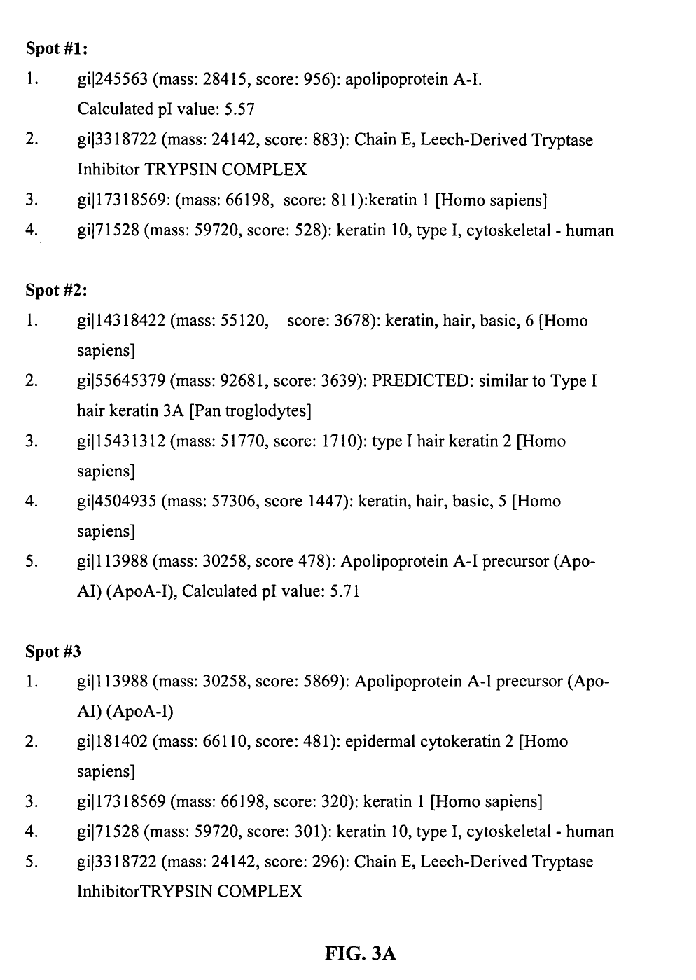

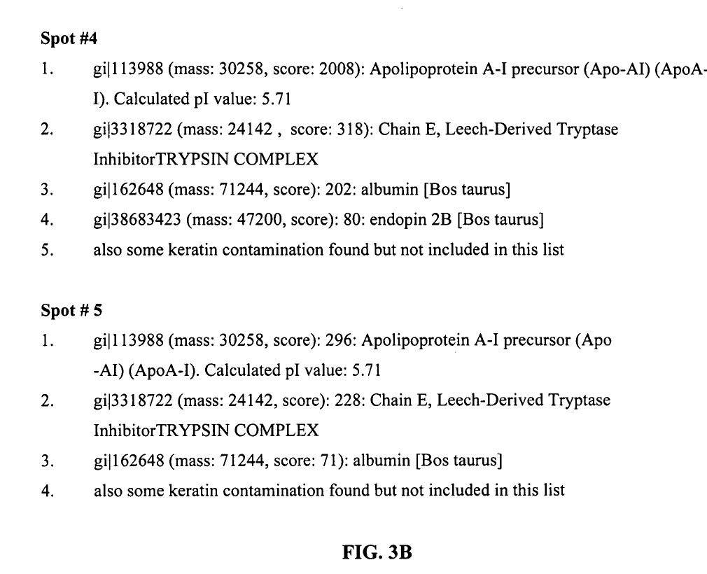

MALDI-TOF-MS protein sequence analysis of 5 spots detected by using streptavidin-HRP and isolated from a 2-D silver-stained gel showed that apolipoprotein A1 and apolipoprotein A1 precursor are the most prominent NVX-207 binding proteins in serum of the cell culture (FIG. 3).

The identification of apolipoprotein (APOA1) in serum as a major NVX-207 binding protein demonstrated a direct link between lipid metabolism and the mechanism of lipid regulation of betulinic acid and its derivatives. For example, APOA1 promotes cholesterol efflux from tissues to the liver for excretion. Apolipoprotein A-I is also the major protein component of high-density lipoprotein (HDL) in the plasma. APOA1 is a cofactor for lecithin cholesterolacyltransferase (LCAT), which is responsible for the formation of most plasma cholesteryl esters. Recently, betulinic acid and other pentacyclic triterpenes were shown to inhibit acyl-CoA:cholesterol acyltransferase (Lee et al., 2006).

### Example 3

This study demonstrated that NVX-207 reduces statin-induced cell death. Induction of cell death was evaluated in a human melanoma cell line (518A2) in the presence of simvastatin and NVX-207 alone or in combination. For these studies, 518A2 cells were treated under normal cell culture conditions in the presence of the compounds at the concentrations indicated in Tables 3, 4, and 5. After 3 days of treatment, cell numbers were counted. Percentages of survival given are cell numbers compared to untreated controls (=100%). Experiments were repeated three times. Mean values are given.

TABLE 3

| NVX-207 (μg/ml) | Survival in % |
|---|---|
| 0.5 | 94 |
| 1.0 | 73 |
| 2.0 | 26 |
| 4.0 | 0 |

TABLE 4

| Simvastatin | |
|---|---|
| (μmol) | Survival (%) |
| 0.5 | 96 |
| 1.0 | 40 |
| 2.0 | 12 |
| 4.0 | 3 |

TABLE 5

| | Survival (%) |
|---|---|
| NVX-207 (0.5 μg/ml) + Simvastatin (μmol) | |
| 1.0 | 66 |
| 2.0 | 39 |
| 4.0 | 24 |
| NVX-207 (1.0 μg/ml) + Simvastatin (μmol) | |
| 1.0 | 63 |
| 2.0 | 46 |
| 4.0 | 31 |

### REFERENCES

The following references, to the extent that they provide exemplary procedural or other details supplementary to those set forth, are expressly and specifically incorporated herein by reference.
U.S. Pat. No. 5,869,535
U.S. Pat. No. 5,962,527
U.S. Pat. No. 6,048,847
U.S. Pat. No. 6,214,814
U.S. Pat. No. 6,228,850
U.S. Pat. No. 6,403,816
US Publn. Appln. 2002091091
US Pubn. Appln. 2002068098
US Pubn. Appln. 2002099164
Bays and Stein, Expert Opin. Pharmacother., 4(11):1901-1938, 2003.
Berg et al., In: Biochemistry, W.H. Freeman and Co., NY, 2002.
CA 2515384
Chong et al., Atherosclerosis, 185(1):21-31, 2006;
DE 19854402
Demierre et al., Nat Rev Cancer, 5(12):930-42, 2005
EP 22-19990922
Gaofu et al., Life Sci., 77(21):2690-2702, 2005.
Grand-Perret et al., Nat. Med., 7(12):1332-1338, 2001.
Harrison's Principles of Internal Medicine, 12^{th} Ed. 1814-1825, McGraw Hill
Harrison's Principles of Internal Medicine, 12^{th} Ed. 1883-1887, McGraw Hill
JP 12-19970331
JP 13-19981006
JP 17-20010619
JP 19-19970603
JP 7-19970311
JP 7-19991026
Kastelein, Int. J. Clin. Pract. Suppl., 134:45-50, 2003.
Lee et al., Biol. Pharm. Bull., 29:382-438, 2006.
Memon et al., Endocrinology, 141(11):4021-4031, 2000.
Myerson et al., J. Lipid. Res., 46(12):2735-2744, 2005.
PCT Appln. 00/03748
PCT Appln. 00/03749
PCT Appln. 00/24762
PCT Appln. 00/26174
PCT Appln. 00/46235
PCT Appln. 00/59492
PCT Appln. 00/59492
PCT Appln. 00/66072
PCT Appln. 00/66080
PCT Appln. 01/17497
PCT Appln. 01/72265
PCT Appln. 01/72315
PCT Appln. 01/90046
PCT Appln. 02/05296
PCT Appln. 02/09719
PCT Appln. 02/09720
PCT Appln. 02/16395
PCT Appln. 02/26761
PCT Appln. 02/26762
PCT Appln. 02/43736
PCT Appln. 02/53138
PCT Appln. 02/78468.
PCT Appln. 02/78685
PCT Appln. 0209698
PCT Appln. 95/04526
PCT Appln. 96/29068
PCT Appln. 9639033
PCT Appln. 98/51293
PCT Appln. 98/51294
PCT Appln. 99/16449
PCT Appln. 99/47113
Phan and Reue, Cell Metab., 1(1):73-83, 2005.
Remington's Pharmaceutical Sciences, 18th Ed. Mack Printing Company, 1990.
Rieusset et al., Mol. Endocrinol., 16(12):2745, 2002.
Shepherd et al., Curr. Med. Res. Opin., 21(5):665-682, 2005.
Sudhop and von Bergmann, Drugs, 62(16):2333-2347, 2002.

What is claimed is:

1. A method of modulating lipid metabolism comprising contacting a cell with betulin, a betulin derivative, betulinic acid, a betulinic acid derivative, or a related steroid-like compound.

2. The method of claim 1, wherein the cell is in a subject.

3. The method of claim 2, wherein the subject is a human.

4. The method of claim 2, wherein the subject has a disorder of intermediate metabolism.

5. The method of claim 1, wherein the cell is contacted with the betulinic acid.

6. The method of claim 1, wherein the cell is contacted with the betulinic acid derivative.

7. The method of claim 6, wherein the betulinic acid derivative has the formula: 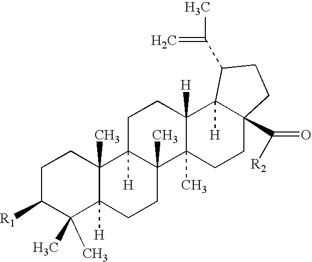
wherein R₁ is a hydroxy group, an amino group, a protected hydroxy group, or a protected amino group; and R₂ is:

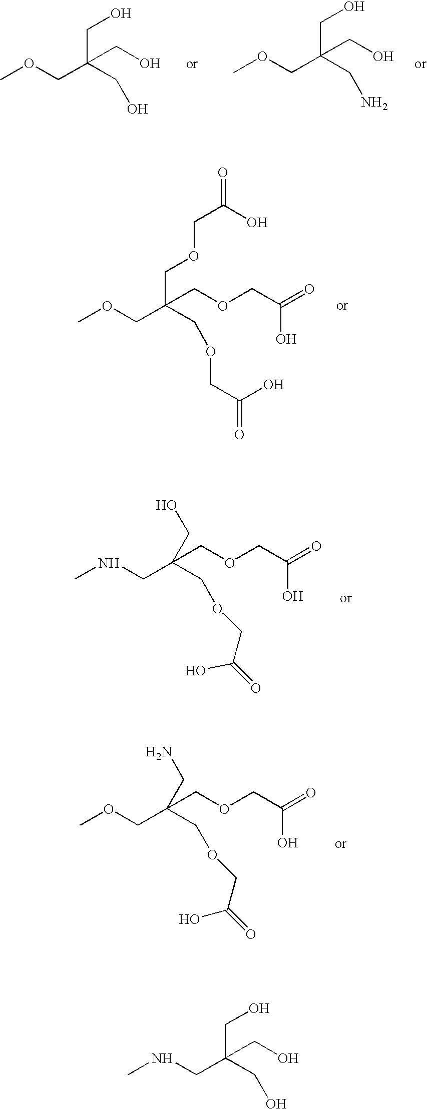

8. The method of claim 6, wherein the betulinic acid derivative is NVX-207.

9. The method of claim 8, further comprising contacting the cell with a statin.

10. A method of treating a subject having a disorder of intermediate metabolism comprising administering to the subject an effective amount of betulin, a betulin derivative, betulinic acid, a betulinic acid derivative, or a related steroid-like compound.

11. The method of claim 10, wherein the subject is a human.

12. The method of claim 10, wherein the subject is administered an effective amount of the betulinic acid.

13. The method of claim 10, wherein the subject is administered an effective amount of the betulinic acid derivative.

14. The method of claim 13, wherein the betulinic acid derivative is NVX-207.

15. The method of claim 10, further comprising administering a second compound that is useful in the treatment of a disorder of intermediate metabolism.

16. The method of claim 15, wherein the second compound is selected from the group consisting of statins, bile acid sequestrants/resins, cholesterol absorption inhibitors, phytostanol analogues, squalene synthase inhibitors, bile acid transport inhibitors, SREBP cleavage-activating protein (SCAP) activating ligands, nicotinic acid (niacin), acipimox, fish oils, antioxidants, policosanol, microsomal triglyceride transfer protein (MTP) inhibitors, acylcoenzyme A: cholesterol acyltransferase (ACAT) inhibitors, gemcabene, lifibrol, pantothenic acid analogues, nicotinic acid-receptor agonists, anti-inflammatory agents, PPAR-alpha agonists, PPAR-gamma agonists, PPAR-alpha/gamma agonisits, PPAR-alpha/gamma/delta agonists, thiazolidinediones (TZD), cholesteryl ester transfer protein (CETP) inhibitors, and synthetic apolipoprotein (Apo)E-related peptides.

17. The method of claim 15, wherein the second compound is a statin.

18. A method of identifying compounds that modulate lipid metabolism comprising:
(a) obtaining a test compound; and
(b) determining whether the test compound has an ability to modulate lipid metabolism in a cell.

19. The method of claim 18, wherein the test compound is betulin, a betulin derivative, betulinic acid, a betulinic acid derivative, or a related steroid-like compound.

20. The method of claim 18, wherein the method of determining is high-throughput screening.

21. The method of claim 18, wherein the cell is in cell culture.

22. The method of claim 18, wherein the cell is comprised in a subject.

23. The method of claim 22, wherein the subject is a mammal.

24. The method of claim 23, wherein the mammal is a human.

25. The method of claim 18 further defined as comprising identifying a compound that modulates lipid metabolism.

26. The method of claim 25, further comprising manufacturing the identified compound.

27. The method of claim 26, further comprising administering the manufactured compound to a subject having a disorder of intermediate metabolism.

28. Use of betulin, a betulin derivative, betulinic acid, a betulinic acid derivative, or a related steroid-like compound for preparation of a medicament for the treatment of a disorder of lipid metabolism.
